(19) United States
(12) Patent Application Publication
(10) Pub. No.: US 2022/0361855 A1
(43) Pub. Date: Nov. 17, 2022

(54) SYSTEM AND METHOD FOR DETERMINING TIME INTERVAL OF FERTILITY

(71) Applicant: AVA AG, Zürich (CH)

(72) Inventors: Raphael GOZLAN, Zürich (CH); Lisa FALCO, Zürich (CH); Franziska DAMMEIER, Zürich (CH)

(21) Appl. No.: 17/753,250

(22) PCT Filed: Aug. 21, 2020
(86) PCT No.: PCT/EP2020/073503

§ 371 Date: Feb. 24, 2022

(30) Foreign Application Priority Data
Aug. 29, 2019 (EP) 19194375.2

(51) Int. Cl. A61B 10/00; A61B 5/00

(52) U.S. Cl. CPC A61B 10/0012; A61B 5/7267; A61B 5/681; A61B 5/74; A61B 2010/0029

(57) ABSTRACT

A method and system are disclosed for determining for a female with irregular menstrual cycles a time interval of fertility, comprising receiving, in a processor (11), from a sensor system (22) of a wearable device (2) of the female, physiological data; determining an estimated time to ovulation of the female, by use of a machine learning model and the physiological data; determining the time interval of fertility using pre-determined time thresholds; and generating a message for the female indicating the time interval of fertility.

### FIELD OF THE INVENTION

The present invention relates to a method and system for determining for a female with irregular menstrual cycles a time interval of fertility. Specifically, the present invention relates to a method, a computer system, and a computer program product for determining for a female with irregular menstrual cycles a time interval of fertility.

### BACKGROUND OF THE INVENTION

The menstrual cycle is governed by a complex interaction of hormones from the ovaries, pituitary gland, and the hypothalamus. On average, the menstrual cycle has a length of 28 days, which can be divided into the follicular phase, ovulation, and the luteal phase.

The menstrual cycle starts with the follicular phase, on the first day of which menstruation occurs. Menstruation is a period of between 2 and 7 days in the follicular cycle in which the uterus sheds its lining. During the follicular phase, follicles mature under the influence of follicle stimulating hormone (FSH), each of them containing a single oocyte. Only one follicle, however, the dominant follicle, will develop until ovulation.

Ovulation is the release of a mature egg from the surface of the ovary and usually occurs mid-cycle. It is a peak in the levels of the luteinizing hormone (LH) that triggers ovulation. The collagenous layers of the follicular wall are proteolytically cleaved, the follicle ruptures, and the ovum is released into the fallopian tubes. The typical lifespan of an egg is only around 24 hours, and unless it is fertilized by a sperm during this time, it will die and conception is no longer possible. Together with the surge of LH, another important hormone, progesterone, starts to rise.

The luteal phase begins after ovulation and lasts approximately two weeks. The ruptured follicle remains on the surface of the ovary and transforms into the corpus luteum. This tissue is characterized by an accumulation of lutein, a yellow carotenoid, and produces progesterone. This hormonal change alters the uterus. Its endometrial lining starts to thicken to prepare the environment for any fertilized egg to attach to the lining, a process called implantation. In the absence of a fertilized egg, progesterone levels fall and the thickened lining of the uterus is shed during the next menses, which marks the start of the next cycle.

Regular menstrual cycles are between 21 and 35 days long with a length that varies by less than 5 days between cycles. Irregular menstrual cycles do not follow this pattern. Females diagnosed with infertility, i.e. who have not gotten pregnant after one year of unprotected intercourse, often have irregular menstrual cycles. The main reasons for menstrual irregularities are hormonal imbalances that prevent ovulation or interfere with the timing of ovulation. Some underlying conditions are known to impair ovulation or its timing, such as thyroid problems, elevated levels of the hormone prolactin, dysfunction of the hypothalamic—pituitary—gonadal (HPG) axis, polycystic ovary syndrome (PCOS), or medication that influences the HPG axis.

As long as the female's cycle is not anovulatory, she can become pregnant even with irregular cycles. However, females with irregular menstrual cycles often face difficulties in conception, as they may have an incorrect assumption of their day of ovulation. Therefore, it may take longer for a woman with irregular menstrual cycles to get pregnant. The use of cycle tracking apps which are only based on the calendar method can be misleading for females with irregular cycles, as they can often wrongly predict ovulation.

Other known methods for trying to conceive for females with irregular cycles are also inaccurate and cumbersome, as they require diligently tracking changes in the mucus and the basal body temperature, using ovulation predictor kits, etc.

### SUMMARY OF THE INVENTION

It is an object of this invention to provide a method and system for determining for a female with irregular menstrual cycles a time interval of fertility. Specifically, the present invention relates to a method, a computer system, and a computer program product for determining for a female with irregular menstrual cycles a time interval of fertility.

According to the present invention, these objects are achieved through the features of the independent claims. In addition, further advantageous embodiments follow from the dependent claims and the description.

According to the present invention, the above-mentioned objects are particularly achieved by a computer-implemented method for determining for a female with irregular menstrual cycles a time interval of fertility, the method comprising receiving, in a processor, from a sensor system of a wearable device of the female, physiological data of the female. The method further comprises determining, by the processor, an estimated time to ovulation of the female, by use of a machine learning model and the physiological data. The method further comprises determining, by the processor, the time interval of fertility using the physiological data, the estimated time to ovulation, and pre-determined time thresholds. The method further comprises generating, by the processor, a message for the female indicating the time interval of fertility.

In an embodiment, receiving the physiological data comprises the processor receiving one or more of: skin temperature data, breathing rate data, resting pulse rate data, heart rate variability data, perfusion data, sleep data, and pulse wave analysis data.

In an embodiment, determining the estimated time to ovulation comprises the processor recording the physiological data for one or more menstrual cycles in a physiological data log and determining the estimated time to ovulation by use of the machine learning model, the physiological data, and the physiological data log.

In an embodiment, determining the estimated time to ovulation comprises the processor training the machine learning model to determine the estimated time to ovulation using machine learning and a training dataset of physiological training data of a large group of females. The person skilled in the art understands that training the machine learning model can be performed on a separate processor and at a time separate from determining the estimated time to ovulation. In particular, the machine learning model can be trained prior to determining the estimated time to ovulation.

In an embodiment, machine learning uses supervised learning.

In an embodiment, determining the estimated time to ovulation of the female comprises the processor using the machine learning model based on a neural network. The machine learning model comprises the neural network.

In an embodiment, determining the estimated time to ovulation comprises the processor using the machine learning model based on one or more specific models and/or methods.

The specific models and/or methods include one of more of the following: a random forest, multiple regressions, logistic multiple regressions, a support vector machine (SVM), and hidden Markov models. The person skilled in the art understands that other types of machine learning model and/or other specific methods can also be used.

In an embodiment, generating the message comprises the processor including in the message an indication of whether the current day is within or outside the time interval of fertility.

In an embodiment, receiving the physiological data further comprises the processor receiving from the female an entry which indicates a time of start of menstruation.

In an embodiment, determining the estimated time to ovulation comprises the processor using the neural network with one or more of: a one-dimensional dilated convolutional layer and a regularization layer.

In an embodiment, determining the estimated time to ovulation comprises the processor using the neural network with two or more stacked one-dimensional dilated convolutional layers. The stacked one-dimensional dilated convolutional layers are modified to be solely retrospective, i.e. causal in that they consider only current and past physiological data, and configured to determine long-term features of an input data sequence of the physiological data, by generating an output using a first one-dimensional dilated convolution layer with a dilation factor one, and using one or more subsequent one-dimensional dilated convolution layers with a dilation factor increased by a factor of two over the previous one-dimensional dilated convolution layer.

In addition to the computer-implemented method for determining for a female with irregular menstrual cycles a time interval of fertility, the present invention also relates to a computer system for determining for a female with irregular menstrual cycles a time interval of fertility, the computer system comprising a processor configured to receive, from a sensor system of a wearable device of the female, physiological data of the female. The processor is configured to determine an estimated time to ovulation of the female, by use of a machine learning model and the physiological data. The processor is configured to determine the time interval of fertility using the physiological data, the estimated time to ovulation, and pre-determined time thresholds. The processor is configured to generate a message for the female indicating the time interval of fertility.

In an embodiment, the processor is configured to receive one or more of: skin temperature data, breathing rate data, resting pulse rate data, heart rate variability data, perfusion data, sleep data, and pulse wave analysis data.

In an embodiment, the processor is further configured to record the physiological data for one or more menstrual cycles in a physiological data log and determine the estimated time to ovulation by use of the machine learning model, the physiological data, and the physiological data log.

In an embodiment, the processor is further configured to train the machine learning model to determine the estimated time to ovulation using machine learning and a training dataset of physiological training data of a large group of females. The person skilled in the art understands that training the machine learning model can be performed on a separate processor and/or at a time separate from determining the estimated time to ovulation. In particular, the machine learning model can be trained prior to determining the estimated time to ovulation.

In an embodiment, machine learning uses supervised learning.

In an embodiment, the processor is further configured to include in the message an indication of whether the current day is within or outside the time interval of fertility.

In an embodiment, the processor is further configured to receive from the female an entry which indicates a time of start of menstruation.

In an embodiment, the processor is configured to use the machine learning model based on a neural network. The machine learning model comprises the neural network.

In an embodiment, the processor is configured to use the machine learning model based on one or more specific models and/or methods. The specific models and/or methods include one of more of the following: a random forest, multiple regressions, logistic multiple regressions, a support vector machine (SVM), and hidden Markov models. The person skilled in the art understands that other types of machine learning model and/or other specific methods can also be used.

In an embodiment, the processor is configured to use the neural network with one or more of a one-dimensional dilated convolutional layer and/or a regularization layer.

In an embodiment, the processor is configured to use the neural network with two or more stacked one-dimensional dilated convolutional layers modified to be solely retrospective and configured to determine long-term features of an input data sequence of the physiological data, by generating an output using a first one-dimensional dilated convolution layer with a dilation factor one, and using one or more subsequent one-dimensional dilated convolution layers with a dilation factor increased by a factor of two over the previous one-dimensional dilated convolution layer.

In addition to the computer-implemented method and the computer system for determining for a female with irregular menstrual cycles a time interval of fertility, the present invention also relates to a computer program product comprising a non-transitory computer-readable medium having stored thereon computer program code configured to control a processor of a computer such that the computer performs the steps receiving physiological data of the female from a sensor system of a wearable device of the female; determining an estimated time to ovulation of the female, by use of machine learning model and the physiological data; determining the time interval of fertility using the physiological data, the estimated time to ovulation, and pre-determined time thresholds; and generating a message for the female indicating the time interval of fertility.

### BRIEF DESCRIPTION OF THE DRAWINGS

The present invention will be explained in more detail, by way of example, with reference to the drawings in which:

FIG. 1: shows a block diagram illustrating schematically a computer system for determining for a female with irregular cycles a time of fertility, and a wearable device.

FIG. 2: shows a block diagram illustrating schematically a computer system for determining for a female with irregular menstrual cycles a time interval of fertility, a wearable device, and a user device.

FIG. 3: shows a flow diagram illustrating a sequence of steps for determining for a female with irregular menstrual cycles a time interval of fertility.

FIG. 4: shows a block diagram illustrating schematically a neural network for determining an estimated time to ovulation using physiological data.

FIG. 5: shows a diagram illustrating schematically stacked dilated one-dimensional convolutional layers.

FIG. 6: shows a time series plot showing how various physiological parameters of a female change during the menstrual cycle.

FIG. 7: shows a time series illustration of how an estimated time to ovulation can vary in relation to time, and how upper and lower thresholds are used to define, using the estimated time to ovulation, days of low fertility and days of high fertility.

### DETAILED DESCRIPTION OF THE PREFERRED EMBODIMENTS

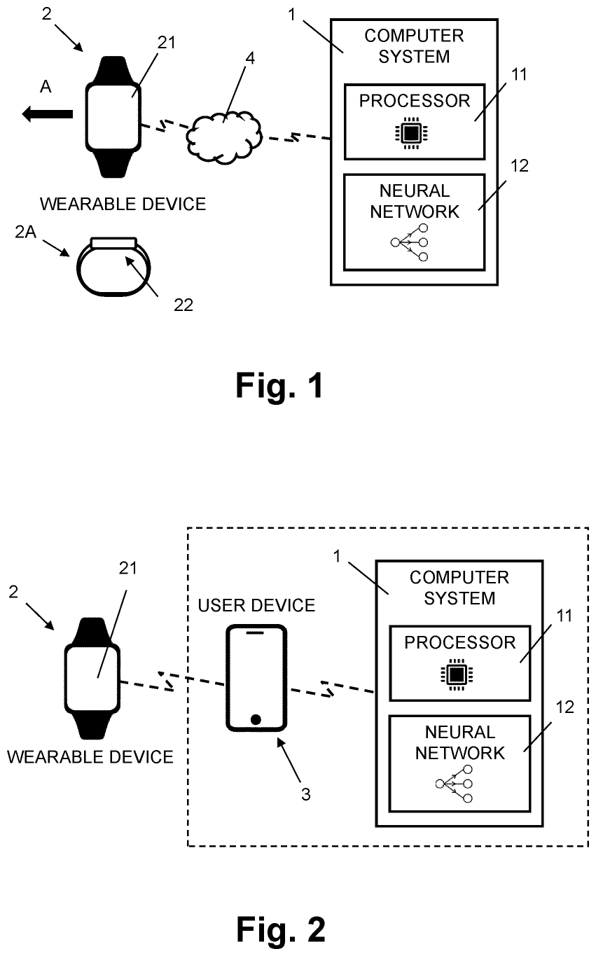

In FIG. 1, reference numeral 1 refers to a computer system comprising one or more computers with one or more processors 11. The computer system 1 can further include various components, such as a data storage system, a communication interface, and/or a user interface. The components of the computer system 1 can be connected to each other via a data connection system, such that they can transmit and/or receive data.

The term data connection system relates to a communication system that facilitates data communication between two components, devices, systems, or other entities. The data connection system can be wired and include a wired connection, such as a cable or system bus. The data connection system can also include wireless communication. The data connection system can also include communication via networks, such as local area networks, mobile radio networks, and/or the Internet 4. The Internet 4 includes, depending on the implementation, intermediary networks.

The processor 11 may comprise a system on a chip (SoC), a central processing unit (CPU), and/or other more specific processing units such as a graphical processing unit (GPU), application specific integrated circuits (ASICs), reprogrammable processing units such as field programmable gate arrays (FPGAs), as well as processing units specifically configured to accelerating certain applications, such as AI (Artificial Intelligence) Accelerators for accelerating neural networks and/or machine learning processes.

The data storage system comprises one or more volatile and/or non-volatile storage components. The storage components may be removable and/or non-removable, and can also be integrated, in whole or in part with the processor 11. Examples of storage components include RAM (Random Access Memory), flash memory, hard disks, data memory, and/or other data stores. The data storage system comprises a non-transitory computer-readable medium having stored thereon computer program code configured to control a processor 11, such that the computer system 1 performs one or more steps and/or functions as described herein. Depending on the embodiment, the computer program code is compiled or non-compiled program logic and/or machine code. As such, the computer system 1 is configured to perform one or more steps and/or functions. The computer program code defines and/or is part of a discrete software application. One skilled in the art will understand, that the computer program code can also be distributed across a plurality of software applications. In an embodiment, the computer program code further provides interfaces, such as APIs, such that functionality and/or data of the computer system 1 can be accessed remotely, such as via a client application or via a web browser.

In an embodiment, the computer system 1 is implemented as a server computer system at a location remote from a female, e.g. in a cloud-based computer system. Reference numeral 12 refers to a neural network. The neural network 12 is a software module which takes as an input physiological data 13 and outputs estimated time to ovulation 14, as explained in more detail below.

A wearable device 2 is worn and/or carried by the female. Reference numeral 2A refers to a cross-sectional view of the wearable device 2 along central axis A. The wearable device 2 comprises a sensor system 22 and a communications module. The sensor system 22 is configured to measure physiological data 13 of the female. The sensor system 22 is preferably entirely integrated into the wearable device 2, however in some embodiments one or more sensor modules of the sensor system 22 may be separate from the wearable device 2 and configured to exchange data with the wearable device 2 in a wired manner, e.g. over a USB (Universal Serial Bus) connection, or wirelessly, e.g. over WLAN (Wireless Local Area Network), Bluetooth, Bluetooth LE (Low Energy), ANT+, mobile radio, etc. For example, the sensor system 22 comprises a first sensor module arranged in the wearable device 2 which is worn as a bracelet on the wrist of the female, along with a second sensor module arranged in a chest-strap worn across the torso of the female. Preferably, the sensor system 22 is in contact with the skin of the female, for example at the wrist, forearm, ankle, fingertip, torso, earlobe, inside the ear, and/or forehead. In an embodiment where the sensor system 22 is integrated directly into the wearable device 2, the wearable device 2 is implemented as appropriate for the part of the body where the sensor system 22 is located. For example, if the sensor system 22 is located at the wrist of the female, the wearable device 2 is implemented as a device worn on the wrist in the form of a bracelet, cuff, or watch, for example the wearable device 2 can be implemented as a health tracker, fitness tracker, or a smart watch. Alternatively, the sensor system 22 is located at the forehead, in which case the wearable device 2 can be implemented as a headband, hat, helmet, etc.

The sensor system 22 is configured to measure physiological data 13, comprising a resting pulse rate, heart rate variability features, blood perfusion, skin temperature, a breathing rate, and/or pulse wave analysis (PWA) data. The physiological data 13 comprises directly measured data and/or data generated by the wearable device 2 from directly measured data. For example, the physiological data 13 comprises both a directly measured time series of physiological signals of the female, as well as data generated therefrom by the wearable device 2, such as maxima, minima, and/or median values.

In a preferred embodiment, the sensor system 22 is configured to acquire a plethysmogram of the female, most preferably a photoplethysmogram (PPG). The physiological data 13 comprises the plethysmogram and/or data generated therefrom. The plethysmograph is used to detect blood volume variations in the microvascular bed of tissue of the female close to the sensor system 22. The sensor system 22 comprises a light source, for example an infrared light or a green light, and one or more photodetectors.

Light shone onto the tissue by the light source penetrates the tissue and is scattered in the tissue such that the one or more photodetectors detect the transmitted and/or reflected light. From the plethysmograph, blood volume changes can be calculated from the amount of detected light.

In a preferred embodiment, an acquired PPG signal from a PPG is transmitted from the sensor system 22 to the wearable device 2. The PPG signal is transmitted over a wired connection, in the case that the sensor system 22 is integrated in the wearable device 2 or connected via a signal line to the wearable device 2, or is transmitted wirelessly, in case the sensor system 22 is arranged in a separate component to the wearable device 2, for example if the wearable device 2 is implemented as a bracelet and the sensor system 22 is arranged on a fingertip of the female.

In an embodiment, the sensor system 22 extracts features from the PPG signal, for example pulse wave analysis (PWA) data. In an example, one or more of the following features are extracted from the PPG signal: a first and/or second derivative, an AC and a DC component, a rise time, an amplitude, a pulse shape, a pulse area, a peak-to-peak interval, a rising edge of the pulse (anacrotic), a falling edge of the pulse (catacrotic), a dictrotic notch, first-order features derived from the first derivate of the PPG signal, as well as further second-order features derived from the second derivative of the PPG signal, such as critical points in the second derivative of the PPG signal located at local maxima, minima, and/or inflection points of the second derivative of the PPG signal. The relationships and ratios between the critical points make it possible to determine a number of health-related parameters and factors. These extracted features are transmitted from the sensor system 22 to the wearable device 2. In an example, the wearable device 2 extracts these features from the PPG signal.

In an embodiment, the wearable device 2 further comprises hardware modules configured to determine a bioelectrical impedance, a thermometer for measuring the temperature of the female, a current time, an acceleration of the wearable device 2, an orientation of the wearable device, a location of the wearable device, and/or ambient conditions, which ambient conditions comprise an air temperature, pressure and/or humidity. The additional determining of the acceleration of the wearable device 2 allows the wearable device 2 to determine movement data of the female, allowing the PPG signal to be processed and interpreted with improved accuracy, as PPG signals can be very susceptible to motion artefacts caused by movement, such as movement of the sensor system 22 by movement of the wrist and/or body of the female during acquiring of the PPG signal. Further, determining the bodily motion of the female allows the wearable device 2 to determine whether the female is motionless or not. This allows the wearable device 2 to determine when measurement data should be acquired and improves the interpretation of the data. For example, resting heart rate is preferably acquired after the female has been at rest for a given period of time, and this can be determined by combining a time of day with movement data. Similarly, a breathing rate is preferably determined after the female has been at rest for a given period of time. In a preferred embodiment, the wearable device 2 measures physiological data 13 at night.

The wearable device 2 comprises a communication interface for wired and/or wireless communication. Wired communication may take place over a Thunderbolt or USB interface, for example. Wireless communication may take place using Bluetooth, Bluetooth LE, Bluetooth Smart, WLAN, ANT+, and/or mobile radio, for example.

In an embodiment, the wearable device 2 further comprises an input module which enables the female to enter additional data into the wearable device 2. For example, the input module comprises a touch interface and/or hardware buttons, such as push-buttons or rotary switches, which input module enables the female to enter additional data into the wearable device 2. The female is enabled to enter additional data such as the specific start of menstruation, the end of menstruation, as well as other data which relate to her menstrual cycle. This additional data is recorded in the wearable device 2 and included as part of the physiological data 13.

In an embodiment, the wearable device 2 further comprises a display module 21. The display module 21 may comprise an electronic display, e.g. an LCD (Liquid Crystal Display), OLED (Organic Light Emitting Diodes), or E Ink display, as well as other hardware elements for visually conveying information to the female, such as LEDs in one or more colors. The touch interface can be integrated into the display module 21.

In an embodiment, the wearable device 2 and the computer system 1 are integrated into a single device, for example a smart-watch.

FIG. 2 shows a block diagram illustrating schematically an embodiment of the invention wherein a user device 3 of the female acts as an intermediary between the wearable device 2 and the computer system 1. The user device 3 is an electronic computing device. For example, the user device 3 is fixedly installed, such as a stationary personal computer, or portable, such as a mobile laptop, tablet, smart phone, or smart watch. The wearable device 2 is connected to the user device 3 using a connection system as described above, for example wired via a USB connection, or wirelessly via Bluetooth. The user device 3 connects to the computer system 1 using a connection system as described above, for example via a mobile radio network, the Internet 4, etc. The user device 3 acts as an intermediary such that the wearable device 2 is enabled to transmit information to the computer system 1 using the user device 3. Similarly, the wearable device 2 receives information from the computer system 1 via the user device 3.

In an embodiment, the user device 3 and the computer system 1 are implemented in a single electronic computing device as described above.

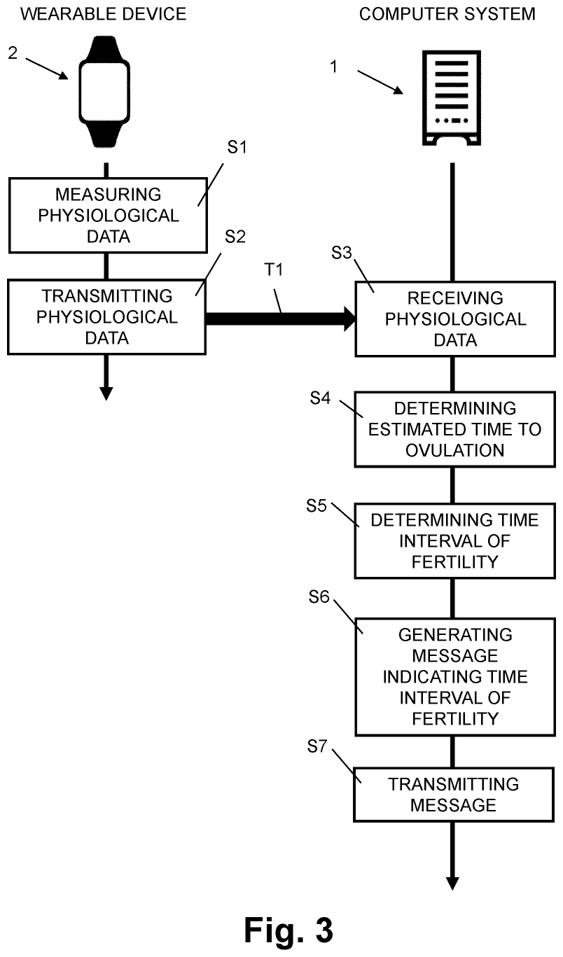

FIG. 3 shows a flow diagram schematically illustrating a sequence of steps for carrying out the invention.

In step S1, the wearable device 2 measures physiological data 13 of the female. The physiological data 13 is processed by the wearable device 2 and/or complemented by additional data entered by the female.

In step S2, the wearable device 2 transmits the physiological data 13, in a transmission T1, from the wearable device 2 to the computer system 1. In particular, the communications module of the wearable device 2 transmits the physiological data 13, via a wired and/or wireless transmission T1, to the processor 11 of the computer system 1, which computer system 1 receives the transmission T1 in step S2. In particular, the processor 11 of the computer system 1 receives the transmission T1 in step S2. Along with the physiological data 13, the wearable device 2 transmits an identifier associated with the female, such as a device identifier and/or a personal identifier.

In an embodiment, the wearable device 2 is implemented as a wearable device worn on the wrist, and the computer system 1 is implemented as a cloud-based computer system 1. Depending on the embodiment and/or configuration, the transmission T1 comprises a wireless transmission to an intermediary device, such as a wireless router or the user device 3 of the female along with a subsequent transmission over the Internet 4 to the computer system 1, and/or the transmission T1 takes place over a mobile radio network. In this embodiment, receiving the transmitted physiological data 13 in a cloud-based computer system 1 has the benefit of allowing the collection of physiological data 13 of large number of females, such that improvements to the functionality of the invention can be undertaken using data analytics, in particular in improving the accuracy of the determined time window of fertility.

In an embodiment where the computer system 1 is implemented in the user device 3 of the female, the transmission T1 is a direct wireless transmission T1 from the wearable device 2 to the computer system 1, e.g. a Bluetooth transmission; alternately, the transmission T1 is an indirect transmission, e.g. via WLAN or a mobile radio network.

In an embodiment where the computer system 1 is integrated into the wearable device 2, the transmission T1 is a wired transmission T1 from the sensor system 22 to the processor 11.

The physiological data 13 transmitted in transmission T1 comprises pulse wave analysis (PWA) data derived from the PPG signal. This PWA data derived from the PPG signal is generated in the wearable device 2 by processing the PPG signal, such that only particular PWA data generated using the PPG signal is transmitted from the wearable device 2 to the computer system 1.

In another embodiment, the physiological data 13 transmitted in transmission T1 comprises the entire measured PPG signal. This enables the computer system 1 to perform data processing on the entire measured PPG signal. This has the advantage of requiring less data processing to occur in the wearable device 2. It also has the advantage of allowing the computationally more powerful computer system 1 to perform the data processing, allowing for a more detailed analysis and comparisons to be made between a large group of females. This has the disadvantage of requiring more data to be transmitted, in the transmission T1, from the wearable device 2 to the computer system 1. Further, it can require more memory in the wearable device 2.

In step S3, the computer system 1 receives the physiological data 13. In an embodiment, the computer system 1 records the physiological data 13 over a period of several days, preferably over one or more menstrual cycles, in a physiological data log.

In step S4, the processor 11 of the computer system 1 determines an estimated time to ovulation 14. The estimated time to ovulation 14 is an interval of time, for instance a number of days, hours, and/or minutes, from a first point in time to a second point in time lying in the future relative to the first point in time, the second point in time being the time point at which ovulation is estimated to occur. The estimated time to ovulation 14 is indicated, in a preferred embodiment, as a number of days. The estimated time to ovulation 14 is determined in the processor 11 using the neural network 12 and the physiological data 13. Specifically, the estimated time to ovulation 14 is determined in the processor 11 using physiological data 13 acquired on the current day, as well as physiological data 13 acquired on one or more past days, preferably a sequence of a number of preceding days. The physiological data 13 is preferably acquired on a given day at night, while the female is asleep. In addition to a current time, in an example physiological data 13 is acquired based on movement data of the female acquired through an accelerometer. This results in more reliable physiological data 13 as the female is at rest during the acquisition and therefore additional context, e.g. if the female is currently doing sport, is not required to interpret the physiological data 13. Using physiological data 13 from a larger number of past days, results in more reliable determination of the estimated time to ovulation 14.

In an embodiment, the computer system 1 uses the physiological data log to generate cycle statistics. The cycle statistics comprise the lengths of previous menstrual cycles of the female, the number of days since the last ovulation, and/or statistical values. The statistical values comprise the minimum, the median, and/or the maximum cycle length of previous menstrual cycles of the female. In addition to the physiological data 13, the cycle statistics can be used to determine the estimated time to ovulation 14.

The determined estimated time to ovulation 14 is on a given day, for example, 8 days until ovulation. In another example, the determined estimated time to ovulation 14 is 6.5 days. The estimated time to ovulation 14 can be determined for the current day, using physiological data 13 acquired on days up to and including the current day. The estimated time to ovulation 14 can also be determined for days in the past. Comparing the estimated time to ovulation 14 determined for days in the past with the estimated time to ovulation 14 determined on the current day enables the computer system 1 to evaluate the precision of the estimated time to ovulation 14. For example, if yesterday the estimated time to ovulation 14 was 8 days, and today the estimated time to ovulation is 7 days, then the precision is high, whereas if today the estimated time to ovulation is also 8 days, or is 6 days, then the precision is lower. Recording the estimated time to ovulation 14 over a number of days is used to improve precision. Further, the onset of menses as entered by the female into the wearable device 2 or the user device 3 is recorded in the data storage system of the computer system 1 to improve the accuracy. The recorded estimated time to ovulation 14 and/or the onset of menses is used, by the computer system 1, to adjust the estimated time to ovulation 14 for improving precision and/or accuracy. The improvements can be limited to the particular female, a group of similar females (e.g. grouped by age, health), or all females.

In an embodiment, the estimated time to ovulation 14 can be a negative time, for example if ovulation was estimated to have occurred on a recent day prior to the current day. As the menstrual cycle is cyclical, the estimated time to ovulation 14 can comprise a plurality of times, for example a negative time representing the estimated time to ovulation 14 of the last ovulation, as well as a positive time representing the estimated time to ovulation 14 of the next ovulation.

In step S5, the processor 11 of the computer system 1 determines the time interval of fertility. The processor 11 uses the determined estimated time to ovulation 14, along with pre-determined thresholds, to determine the time interval of fertility. The time interval of fertility is a span of time during the menstrual cycle, for example a sequence of days, within which the female is considered to be fertile and within which unprotected intercourse may lead to conception. The pre-determined thresholds comprise an upper threshold and a lower threshold. If, for a given day, the estimated time to ovulation 14 falls between the upper threshold and the lower threshold, then the given day is considered to be a day of high fertility and therefore within the time interval of fertility. If, for a given day, the estimated time to ovulation 14 is higher than the upper threshold or lower than the lower threshold, then the given day is considered to be a day of low fertility and therefore not within the time interval of fertility. The processor 11 determines, for a given day, which days in the future, relative to the given day, lie in the time interval of fertility. The given day is the current day, or a day in the past.

In an embodiment, the pre-determined thresholds are a threshold function. The threshold function is configured such that the processor 11 of the computer system 1 determines the time interval for fertility, using as input the estimated time to ovulation 14.

In an embodiment, the threshold function is configured such that the processor 11 determines whether a given day lies within or outside the time interval of fertility. The threshold function is further configured such that the processor 11 categorizes the given day into more than two categories, for example into the categories of: low fertility, medium fertility, and high fertility. Additionally, the processor 11 determines a likelihood of a pregnancy occurring on a given day.

In an embodiment, the pre-determined thresholds comprise two functions, an upper threshold function and a lower threshold function. The upper threshold function is configured such that the processor 11 determines, using the estimated time to ovulation 14, a first point in time when the time interval of fertility opens. In particular, the time interval of fertility opens when the estimated time to ovulation 14 falls below a first value. The first value is between three (3) days and seven (7) days, preferably between four (4) and five (5) days, most preferably four and a half (4.5) days. The lower threshold function is configured such that the processor 11 determines, using the estimated time to ovulation 14, a second point in time when the time interval of fertility closes. In particular, the time interval of fertility closes when the estimated time to ovulation 14 falls below a second value. The second value is between zero (0) and minus two (−2) days, preferably minus one (−1) day. In the cases that the second value is negative, the one or more days after ovulation is estimated to have occurred lie within the time interval of fertility.

In an embodiment, other values derived from the estimated time to ovulation 14 may also be used by the threshold function or the upper and/or the lower threshold functions for determining the time interval of fertility. These other values are one or more of the following: a rate of change of the estimated time to ovulation 14 over a given time interval and/or an area under the curve of the estimated time to ovulation 14 over a given time interval.

In an embodiment, the values of the upper threshold and the lower threshold are values specific to the female.

In an embodiment, the upper and lower threshold functions are configured such that the processor 11 determines whether the current day lies within or outside the time interval of fertility. The upper and lower threshold functions are configured such that the processor 11 determines the likelihood of conception on a given day.

In step S6, the processor 11 of the computer system 1 generates a message for the female comprising the time interval of fertility. The message can further comprise an identifier associated with the female and/or a device identifier or communication address associated with the wearable device 2 of the female, such that the message can be addressed to the correct female and/or user device 3 of the female.

In step S7, using the communication module of the computer system 1, the processor 11 of the computer system 1 transmits the message from the computer system 1 to the wearable device 2. The wearable device 2 indicates to the female the time interval of fertility.

In an embodiment, the wearable device 2 indicates whether the current day is within the time interval of fertility, and/or the likelihood of conception on the current day.

In an embodiment, the processor 11 of the computer system 1 transmits the message from the computer system 1 to the user device 3 of the female. The user device 3 receives the message and indicates to the female the time interval of fertility, using a display of the user device 3. The user device 3 can further indicate whether the current day lies within or outside the time interval of fertility and/or the likelihood of conception on the current day. In one example, where the user device 3 is implemented as a smart phone, the smart phone comprises an application which receives the message from the computer system 1. In another example, where the user device 3 is implemented as a personal computer, the personal computer receives the message, for example via a web-browser.

Displaying the time interval of fertility comprises displaying one or more days and indicating, for example, whether the one or more days are in the time interval of fertility or not. In particular, displaying the time interval of fertility comprises displaying whether the current day is in the time interval of fertility.

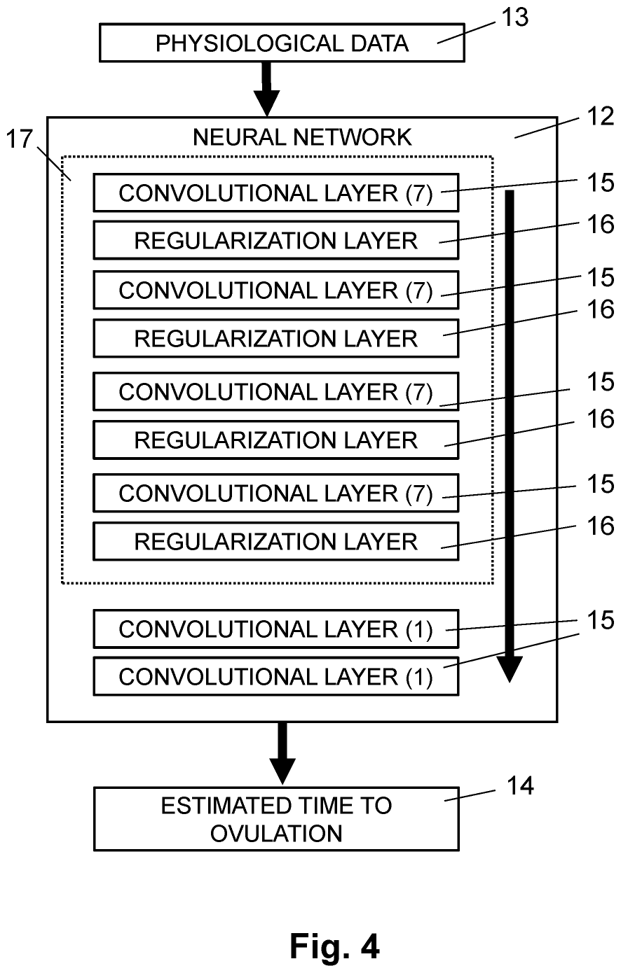

s FIG. 4 illustrates schematically the neural network 12. Physiological data 13 is input into the neural network 12, which is processed by the neural network 12 to determine the estimated time to ovulation 14. In particular, the neural network 12 is configured such that the processor 11 of the computer system 1, determines the estimated time to ovulation 14 using physiological data 13. The neural network 12 is a neural network which implements a function which can be decomposed into other functions. The neural network 12 comprises a set of parameters. The neural network 12 can be represented as a sequence of layers, which layers may be grouped into sub-networks. Each layer comprises a set of nodes, each node having adjustable parameters including at least one or more weight parameters and a bias parameter. The sequence of layers comprises an input layer, into which physiological data 13 is passed, a succession of hidden layers, and an output layer, which outputs the estimated time to ovulation 14. The hidden layers of the neural network 12 comprise one or more of the following types: a one-dimensional convolutional layer 15 and/or a regularization layer 16. The one-dimensional convolutional layers 15 are configured to learn short-term and long-term features of the physiological data 13. The regularization layers 16 are configured to reduce overfitting of the neural network 12 during training. In the context of this invention a regularization layer 16 means that the layer preceding the regularization layer 16 is subject to regularization during training of the neural network 12.

In an embodiment, the regularization layers 16 are implemented to reduce the likelihood of overfitting the neural network 12 during training.

In an embodiment, the one-dimensional convolutional layers 15 are one-dimensional dilated convolutional layers 15, with a kernel size k and a dilution factor d. One-dimensional dilated convolutional layers 15 in a sequence are called stacked one-dimensional convolutional layers 15, as explained in more detail below. The neural network 12 comprises a stack 17 comprising stacked one-dimensional dilated convolutional layers 15. The kernel size k is between two (2) and fourteen (14) days, preferably between four (4) and ten (10) days, most preferably seven (7) days.

The stack 17 is connected to one or more downstream convolutional layers 15. The final convolutional layer 15 outputs the estimated time to ovulation 14.

The neural network 12 is initially in an untrained state. The neural network 12 is then trained in the training phase, which takes place before the neural network 12 is used to determine the estimated time to ovulation 14 based on physiological data 13 of the female.

The neural network 12 is trained to determine the estimated time to ovulation 14 using machine learning and a training dataset of physiological training data of a large group of females with irregular menstrual cycles. The neural network 12 is trained using supervised learning, in which labelled physiological data is used to optimize the parameters of the neural network 12.

In particular, the physiological training data comprises labelled physiological data of the menstrual cycle from a large group of females with irregular menstrual cycles labelled with an ovulation day. The labelled physiological data is used during training of the neural network 12.

The neural network 12 is trained by determining the estimated time to ovulation 14 for a given day using the labelled physiological data. To improve the accuracy of the neural network 12, the determined estimated time to ovulation 14 is compared to the actual time to ovulation as specified in the labelled physiological training data used for training. In particular, a loss function is used. For example, the loss function comprises an L1 and/or L2 error term, as is commonly used in the art of neural networks for regression. The goal of training the neural network 12 is to iteratively minimize the loss function using a first order optimization technique and back-propagation. Successive training iterations result in a neural network 12 with determined estimated times to ovulation 14 lying closer and closer to the actual time to ovulation as specified in the labelled physiological data, as reflected mathematically by a smaller L1 and/or L2 error term of the loss function.

The neural network 12 is stored, by the processor 11, in the data storage system of the computer system 1, for use in determining the time to ovulation 14 of the female.

The training of the neural network 12 takes place using the processor 11 of the computer system 1. However, in an embodiment, this takes place on a separate computer system 1, such as a remote cloud-computing server, after which the neural network 12 is transmitted to the processor 11 of the computer system 1. In an embodiment, the neural network 12 is further transmitted to the wearable device 2 or the user device 3 of the female.

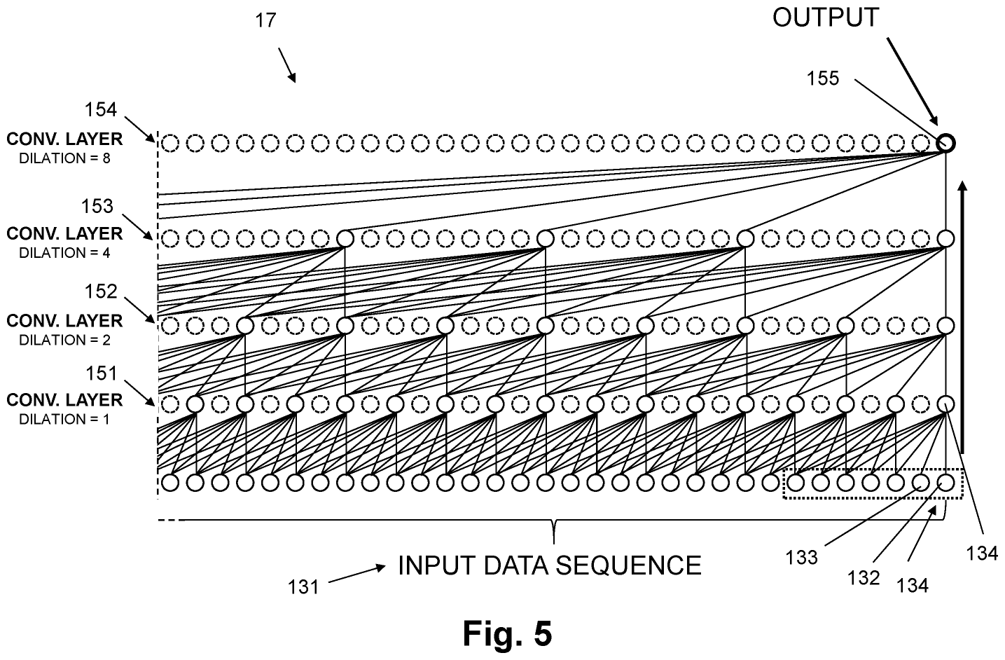

FIG. 5 illustrates schematically a stack 17 comprising four one-dimensional dilated convolutional layers 15 stacked such that the output of the first layer is input into the second layer and so on. An input data sequence 131 is generated by the processor 11 using the physiological data 13 from a sequence of days, illustrated as a row of nodes each comprising physiological data 13 from a given day, with the current day on the right side and past days arranged in a sequence from the current day to the left. To avoid clutter in the illustration, only those connections are shown between the nodes of the layers which are relevant for determining an output 155 of the current day. For determining outputs of prior days, the connections are shifted to the left by the same number of days.

The input data sequence comprises current physiological data 132 from the current day, past physiological data 133 from a day prior to the current, and further physiological data 13 from other past days of the current and past menstrual cycles.

A first one-dimensional dilated convolutional layer 151 has a kernel size k of seven (7), as illustrated by the dotted rectangle 134, and a dilation factor d of one (1), and therefore receives as an input to an input node 134 current physiological data 132 as well as data from six (6) days prior to the current day. The dilation factor d of one (1) means that each node from a prior layer is used as an input to the current one-dimensional dilated convolutional layer 151. The first one-dimensional dilated convolutional layer 151 therefore learns short-term features of the input data sequence 131, in particular it learns features and/or patterns which are present when considering seven days up to and including a given day. It therefore has a receptive field of seven (7) days.

A second one-dimensional dilated convolutional layer 152 has a kernel size k of seven (7), and a dilation factor d of two (2), and therefore skips every second output from the first one-dimensional dilated convolutional layer 151. Therefore, the second one-dimensional dilated convolutional layer 152 learns features and/or patterns present in nineteen (19) days, and therefore has a receptive field of nineteen (19) days.

In a similar manner, a third one-dimensional dilated convolutional layer 153 has a kernel size k of seven (7) and a dilation factor d of four (4), such that it has a receptive field of forty-three (43) days, and a fourth one-dimensional dilated convolutional layer 154 has a kernel size k of seven (7) and a dilation factor d of eight (8), such that it has a receptive field of ninety-one (91) days.

In general, the size RF of the receptive field of a stack of n one-dimensional dilated convolutional layers with kernel size k and dilation factors growing by an exponent of two (2) (i.e. the sequence of dilation factors 1, 2, 4, 8, . . . ) equals

RF=(k−1)×(2ⁿ−1)+1.

The stack 17 of one-dimensional dilated convolutional layers 15 allows the neural network 12 to learn features in the input data sequence 131 of a plurality of irregular menstrual cycles of the female. The benefits of using dilated layers is that less connections are required than if the layers were not connected, and therefore that the training and storage of the neural network 12 is more computationally efficient, and that each layer is configured to learn features present in a receptive field of a particular size and therefore is configured to learn abstractions at a certain time-scale. For example, the first one-dimensional convolution layer 151 can learn short-term features of the input data sequence 131, such as if a particular physiological value in the physiological data 13 is increasing or decreasing. The second one-dimensional convolution layer 152 can learn more abstract patterns or a combination of lower level patterns using the output of the first one-dimensional convolution layer 151 with its receptive field.

Typically, in the art of neural networks, one-dimensional convolutional layers 15 are configured such that short-term features of the sequence are generated by considering both sequence terms before and after a given day, i.e. the kernel is arranged symmetrically around the given day and therefore the one-dimensional convolutional layer 15 is both preemptive and retrospective. However, the one-dimensional convolutional layers 15 according to the invention are configured such that they are solely retrospective, as this relates to how the data is acquired and because the invention relates to the prediction of the time interval of fertility.

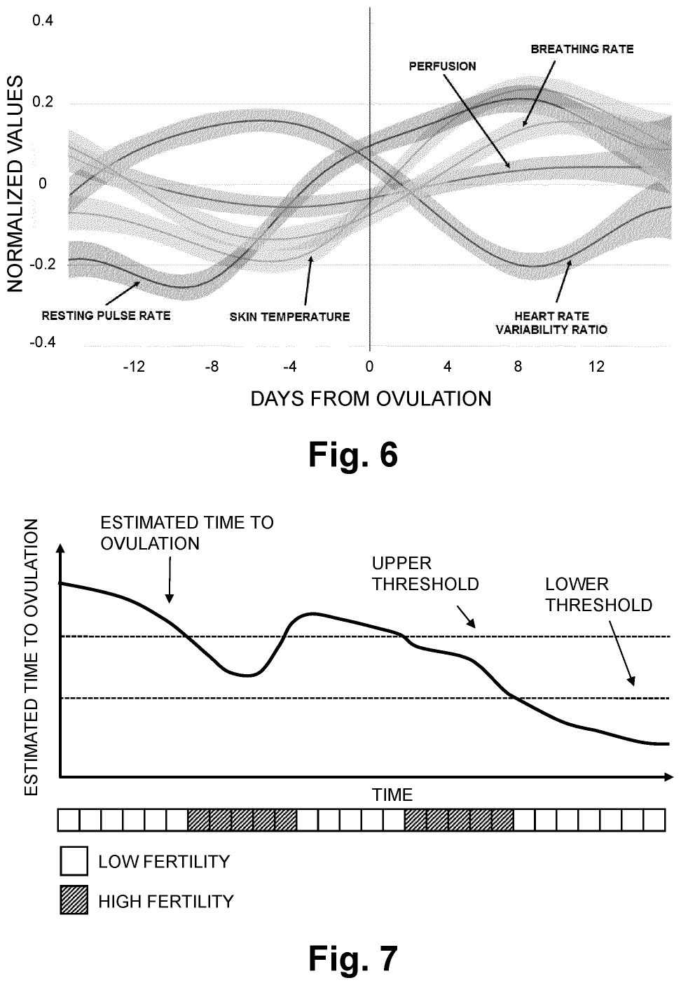

FIG. 6 shows a time series plot illustrating how various physiological parameters of a female change during the menstrual cycle, the physiological parameters being comprised in the physiological data 13. In particular, it shows how normalized values of perfusion, breathing rate, resting pulse rate, skin temperature, and heart rate variability ratio depend on the number of days from ovulation. Analytically determining how these physiological parameters are related to the day of ovulation and therefore how they relate to the time interval of fertility for females with irregular menstrual cycles is very complex. However, using the neural network 12 with the particular architecture as described above, a physiological training dataset comprising physiological training data from a very large dataset of females, and an appropriate training method (i.e. defining an appropriate loss function), parameters of the neural network 12 can be determined such that the neural network 12 can reliably determine the estimated time to ovulation 14 using the physiological data 13, and from that the time interval of fertility, using the thresholds.

FIG. 7 shows a time series illustration of how the estimated time to ovulation 14 changes during an example of an irregular menstrual cycle and how this estimated time to ovulation 14 is used along with the upper and lower thresholds to define the time interval of fertility, which is comprised of days of low fertility (as indicated by white squares in FIG. 7) and days of high fertility (as indicated by hatched squares).

It can be seen that the estimated time to ovulation 14 varies throughout the menstrual cycle, beginning with a high estimated time to ovulation 14, which decreases until the estimated time to ovulation 14 is lower than the upper threshold. The day on which this occurs is designated a day of high fertility. If the estimated time to ovulation 14 increases above the upper threshold, the day on which this occurs is designated a day of low fertility. The estimated time to ovulation 14 determined by the neural network 12 can also be negative, and the lower threshold is, in an embodiment, a negative value. The estimated time to ovulation 14 is negative when the neural network 12 determines that ovulation has occurred in the past.

It should be noted that, in the description, the sequence of the steps has been presented in a specific order, one skilled in the art will understand, however, that the order of at least some of the steps could be altered, without deviating from the scope of the invention.

1. A method for determining for a female with irregular menstrual cycles a time interval of fertility, the method comprising:
receiving, in a processor, from a sensor system of a wearable device of the female, physiological data of the female;
determining, by the processor, an estimated time to ovulation of the female, by use of a machine learning model and the physiological data;
determining, by the processor, the time interval of fertility using the physiological data, the estimated time to ovulation, and pre-determined time thresholds; and
generating, by the processor, a message for the female indicating the time interval of fertility.

2. The method according to claim 1, wherein receiving the physiological data comprises the processor receiving one or more of: skin temperature data, breathing rate data, resting pulse rate data, heart rate variability data, perfusion data, sleep data, and pulse wave analysis data.

3. The method according to claim 1, wherein determining the estimated time to ovulation comprises the processor:
recording the physiological data for one or more menstrual cycles in a physiological data log; and
determining the estimated time to ovulation by use of the machine learning model, the physiological data, and the physiological data log.

4. The method according to claim 1, wherein determining the estimated time to ovulation comprises the processor training the machine learning model to determine the estimated time to ovulation using machine learning and a training dataset of physiological training data of a large group of females.

5. The method according to claim 1, wherein determining the estimated time to ovulation of the female comprises the processor using the machine learning model based on a neural network.

6. The method according to claim 5, wherein determining the estimated time to ovulation comprises the processor using the neural network with one or more of: a one-dimensional dilated convolutional layer and a regularization layer.

7. The method according to claim 5, wherein determining the estimated time to ovulation comprises the processor using the neural network with two or more stacked one-dimensional dilated convolutional layers modified to be solely retrospective and configured to determine long-term features of an input data sequence of the physiological data, by generating an output using a first one-dimensional dilated convolution layer with a dilation factor one, and using one or more subsequent one-dimensional dilated convolution layers with a dilation factor increased by a factor of two over the previous one-dimensional dilated convolution layer.

8. A computer system for determining for a female with irregular menstrual cycles a time interval of fertility, the computer system comprising a processor configured to:
receive, from a sensor system of a wearable device of the female, physiological data of the female;
determine an estimated time to ovulation of the female, by use of a machine learning model and the physiological data;
determine the time interval of fertility using the physiological data, the estimated time to ovulation, and pre-determined time thresholds; and
generate a message for the female indicating the time interval of fertility.

9. The computer system according to claim 8, wherein the processor is configured to receive one or more of:
skin temperature data, breathing rate data, resting pulse rate data, heart rate variability data, perfusion data, sleep data, and pulse wave analysis data.

10. The computer system according to claim 8 or 9, wherein the processor is further configured to:
record the physiological data for one or more menstrual cycles in a physiological data log; and
determine the estimated time to ovulation by use of the machine learning model, the physiological data, and the physiological data log.

11. The computer system according to claim 8 or 9, wherein the processor is further configured to train the machine learning model to determine the estimated time to ovulation using machine learning and a training dataset of physiological training data of a large group of females.

12. The computer system according to claim 8 or 9, wherein the processor is configured to use the machine learning model based on a neural network.

13. The computer system according to claim 12, wherein the processor is configured to use the neural network with one or more of:
a one-dimensional dilated convolutional layer and a regularization layer.

14. The computer system according to claim 12, wherein the processor is configured to use the neural network with two or more stacked one dimensional dilated convolutional layers modified to be solely retrospective and configured to determine long-term features of an input data sequence of the physiological data, by generating an output using a first one dimensional dilated convolution layer with a dilation factor one, and using one or more subsequent one-dimensional dilated convolution layers with a dilation factor increased by a factor of two over the previous one-dimensional dilated convolution layer.

15. A computer program product comprising a non-transitory computer-readable medium having stored thereon computer program code configured to control a processor of a computer such that the computer performs the steps:
receiving physiological data of the female from a sensor system of a wearable device of the female;
determining an estimated time to ovulation of the female, by use of a machine learning model and the physiological data;
determining the time interval of fertility using the physiological data, the estimated time to ovulation, and pre-determined time thresholds; and
generating a message for the female indicating the time interval of fertility.
